(19) United States
(12) Patent Application Publication
(10) Pub. No.: US 2010/0090004 A1
(43) Pub. Date: Apr. 15, 2010

(54) SYSTEM AND METHOD FOR INVENTORY MANAGEMENT OF MEDICAL ASSETS

(76) Inventors: Daniel L. Sands, Warsaw, IN (US); Matthew R. LaFontaine, Fort Wayne, IN (US); Court H. Sailor, Spencerville, IN (US); Michael D. Westrick, Huntertown, IN (US)

Correspondence Address: TAYLOR & AUST, P.C., P.O. Box 560, 142. S Main Street, Avilla, IN, 46710, US

(21) Appl. No.: 12/576,324

(22) Filed: Oct. 9, 2009

Related U.S. Application Data
(60) Provisional application No. 61/104,018, filed on Oct. 9, 2008.

(51) Int. Cl. G06Q 90/00

(52) U.S. Cl. 235/385

(57) ABSTRACT

A system for identification, tracking and management of medical assets, includes a plurality of RFID tagged medical assets, and a completely networked supply chain. The supply chain includes at least one end user location; at least one distributor location; an offsite data management system; a plurality of RFID readers; and at least one data network. Each RFID reader is interspersed at a respective location throughout the networked supply chain and configured to electronically read data from the plurality of RFID tagged medical assets. The one or more data networks interconnect each end user location, each distributor location, and the offsite data management system. The one or more data networks provide real time information from at least one of the plurality of RFID readers to the offsite data management system concerning a current attribute associated with at least one of the RFID tagged medical assets.

### CROSS REFERENCE TO RELATED APPLICATIONS

This is a non-provisional application based upon U.S. provisional patent application Ser. No. 61/104,018, entitled “RFID FEASIBILITY ANALYSIS”, filed Oct. 9, 2008, which is incorporated herein by reference.

### BACKGROUND OF THE INVENTION

1. Field of the Invention

The present invention relates to a system for identification, tracking and management of RFID tagged medical assets.

2. Description of the Related Art

Several medical device segments, including orthopedics, cardiovascular, oncology and materials management functions within hospitals have significant labor inefficiencies and excess inventory within their logistic processes that can benefit from radio frequency identification (RFID) and related software technology. As an example, orthopedic implant original equipment manufacturers (“OEMs”) and their network of independent stocking distributors manage $11 billion of worldwide implant and surgical instrument inventory to ensure that the right orthopedic implants and their associated surgical instruments are on hand for surgeries when scheduled. Due to rapidly changing implant technology and continual improvements in surgical instruments, OEMs and distributors consign and loan implants and related surgical instrument kits and cases to hospitals, thereby retaining ownership of these high value assets while they are deployed throughout the world. Lacking a reliable, accurate inventory method to track these assets throughout the supply chain, but wanting to avoid a negative medical experience at all costs, OEMs and distributors deploy expensive excess inventory and field personnel to ensure the appropriate implants and instruments are available at point of use as required. The industry has as much as 60% redundant inventory, or $6.5 billion of excess cost built into OEM and distributor operations.

Typically, manual inventory audits within an Orthopedic distributor territory and hospital are conducted by the OEM annually which is time consuming and costly. Due to the infrequent nature of audits, the information becomes outdated almost instantly and typically is only entered into the OEM's enterprise resource management system. Each Distributor may have a database of inventory it is managing for its territory, but typically there is no connectivity or common location of this data that is accessible to the manufacturer or hospital. To compound the problem, hospitals may consign OEM inventory but not put any inventory data into their Materials Management software system. Because of the high volume and frequent movement of product from distributor to distributor or from hospital to hospital, traceability becomes very labor intensive. Some bar code scanning of labeled sterile implants occurs, but generally the sales reps moving the product are not affective at this task. The surgical instrument cases and instruments typically do not have any bar code or unique identification for traceability purposes.

Other RFID technologies have been attempted in the orthopaedic space such as LF passive, and HF passive and active technology. Their limitations are limited read distance and proprietary reader networks are required and they are not globally interoperable due to limited international standards.

### SUMMARY OF THE INVENTION

The present invention provides a unique identification on product that is readable by standard reader technology and an automated way to capture the movement of orthopaedic product from manufacture to distributor to hospital and post the information regarding its location to a single data repository for accessibility via the world wide web.

The present invention also provides a system that includes the combination of RFID medical grade tags, RFID hardware, hardware middleware, an RFID scan engine software and database and customizable workflow application software that automatically compiles data from RFID tag reads (unique product identification, time and date stamp) from networked RFID readers that automatically read the tag ID from the unique IP address of each of the networked readers. This raw read data is filtered into a common database. This data can be accessible via the world wide web.

The invention in one form is directed to a system for identification, tracking and management of medical assets, including a plurality of RFID tagged medical assets, and a completely networked supply chain. The supply chain includes at least one end user location; at least one distributor location; an offsite data management system; a plurality of RFID readers; and at least one data network. Each RFID reader is interspersed at a respective location throughout the networked supply chain and configured to electronically read data from the plurality of RFID tagged medical assets. The one or more data networks interconnect each end user location, each distributor location, and the offsite data management system. The one or more data networks provide real time information from at least one of the plurality of RFID readers to the offsite data management system concerning a current attribute associated with at least one of the RFID tagged medical assets.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawing, wherein:

FIG. 1 is a graphical illustration of an embodiment of the system of the present invention for the identification, tracking and management of medical assets;

FIG. 2 is a perspective view of a portion of an embodiment of an RFID tagged surgical case which may be used with the system of the present invention;

FIG. 3 is a perspective view of an embodiment of a portal type RFID reader which may be used with the system of the present invention; and

FIG. 4 is a perspective view of an embodiment of a smart cabinet containing RFID tagged medical assets and used with the system of the present invention.

Corresponding reference characters indicate corresponding parts throughout the several views. The exemplification set out herein illustrates an embodiment of the invention, in one form, and such exemplification is not to be construed as limiting the scope of the invention in any manner.

### DETAILED DESCRIPTION OF THE INVENTION

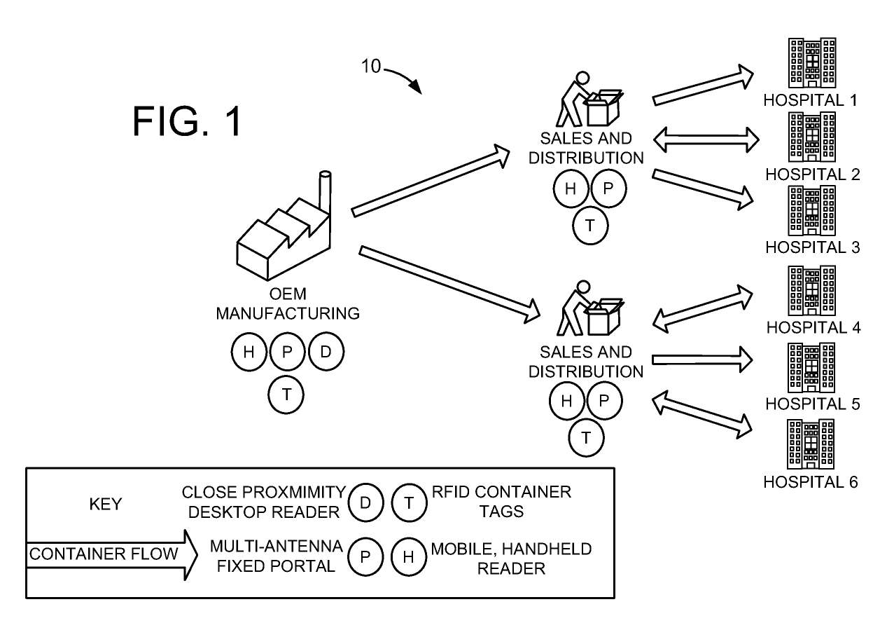

Referring now to FIG. 1, the system 10 of the present invention includes 1.) medical grade autoclavable Ultra High Frequency (UHF) RFID tags that survive sterile reprocessing and perform in, on or around metal at a read distance ranging from 6 in. for item level instruments to 15 feet for case level or package level tracking; 2.) UHF RFID label inlays are typically applied to implant packaging; 3.) RFID tags or labels are encoded with a unique Identification number that is associated with the OEM's product; 4.) RFID handheld mobile readers and RFID stationary readers are strategically positioned within “choke points” or common work or product flow points within the OEM's distribution network; 5.) The RFID readers are controlled by a proprietary scan engine that manages each reader and its antenna and which has incorporated logical statements within the software that filter and disseminate the unique RFID code that is commissioned to each product in the OEM's supply chain. Furthermore, each reader location has a unique internet protocol address to associate to a given location within the OEM's distribution network; 6.) RFID tag reads or data is stored and manipulated within an offsite database consisting of stored procedures and fields organized in a manner consistent with the workflow of the OEM; 7.) Software graphical user interfaces may exists at each user workstation that is unique to the OEM or that comes standard and is accessible via the world wide web which provides basic inventory reporting and product read location transaction histories.

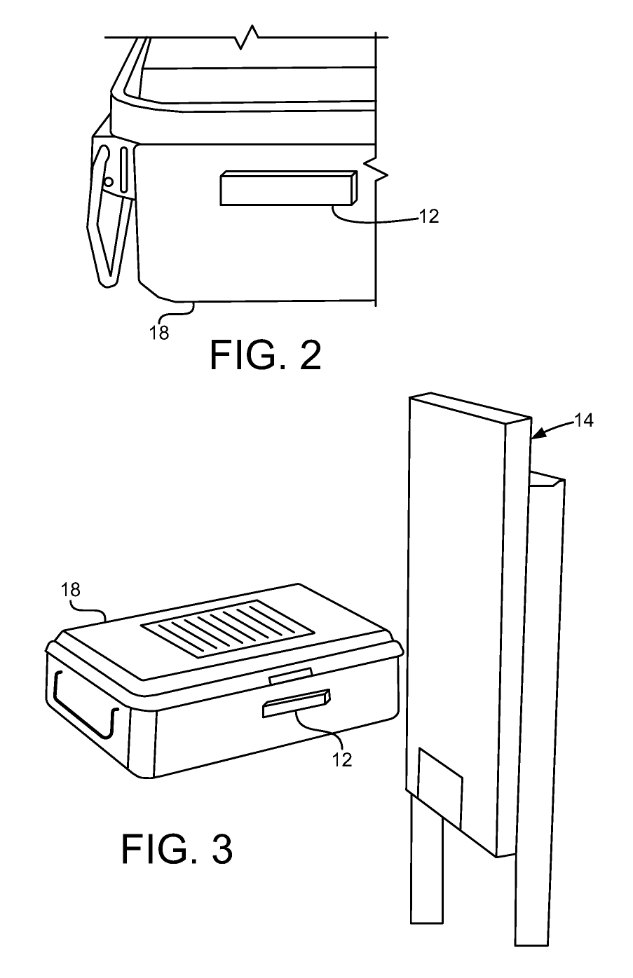

UHF Generation 2 is a global band standardized technology that is interoperable among the various reader and tag manufacturers. The flexibility of UHF technology provides a low cost disposable label for non-detuning applications such as cardboard packaging. The presence of metal, liquid and heat may detune normal UHF labels. However, the present invention utilizes very small, durable reusable rigid medical grade tags 12 (FIGS. 2 and 3) that make extended read distance possible in more RF unfriendly environments. This flexibility in RFID tags enables that one reader infrastructure can be deployed making it less expensive and more easy to maintain over time.

The present invention is a comprehensive, customizable, turn-key asset and inventory management system, consisting of RFID and software technologies. The present invention tracks high-value medical devices and people and automates critical healthcare workflow processes ensuring that the right products and people are in the right place at the right time for hospitals, medical device manufacturers and their distributor agents. The system creates value by eliminating the need for high-value excess inventory, reducing field personnel tasked with manual inventory management, providing remote inventory visibility, reducing labor and improving patient safety by automating work flow processes and ensuring compliance with medical device FDA regulations, thereby potentially saving medical device companies and care facilities within the supply chain millions of wasted dollars.

The present invention automatically records “read transactions” as tagged devices or people come in range of strategically placed readers throughout the supply chain. These automatic read transactions generate time and location data that are stored in a common data repository, and create the shared real time “last seen” visibility necessary to reduce inventory and labor costs for medical device manufacturers and hospitals.

The present invention is scalable and designed to ensure surgical readiness; work across multiple OEMs and supplier networks; work in the medical device physical environment and ensure functionality, dependability and durability; and work across all supply chain points by offering systems designed to meet healthcare industry standards. The completely networked data network may include a local area network (LAN), a wide area network (WAN) and/or a cellular telephone connection.

The products and services of the present invention include:
Custom RFID tag engineering
System engineering
Medical Grade and harsh environment RFID Tags
RFID Hardware (reader/writer devices, antenna)
Middleware Software
System Installation and Data Integration
System training and maintenance

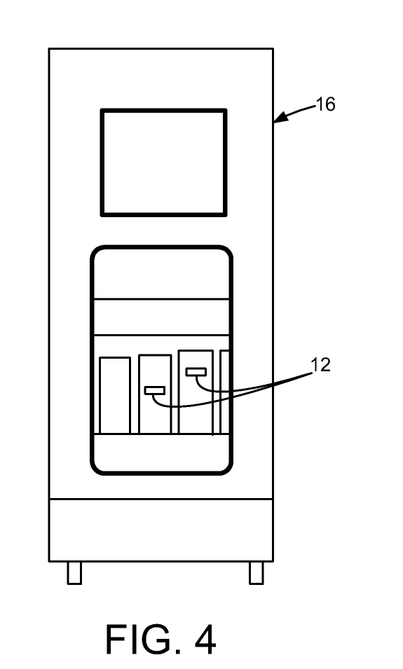

The present invention provides both hardware and software throughout an OEM's supply chain matching their common workflow or “choke points”. The readers or read locations can be configured as fixed portal readers 14 (FIG. 3) or a “smart cabinet” reader 16 (FIG. 4; described in more detail below). Each system is fitted to the particular physical sites (hardware location/mounting) and resident Enterprise Resource Planning (ERP) systems (interface of scan engine for RFID-generated data into resident ERP systems).

Medical Grade UHF RFID tags are read by multiple UHF RFID manufacturers' reader and antenna systems in the ultra high frequency (UHF) spectrum allowing global and multi-location standard readability. The present invention can approach universal readability by creating and customizing RFID tags in various protective form factors to survive the medical device environment, utilizing best of class readers and antenna instead of proprietary platforms. Internet based tracking software addresses the dynamic need for a centralized data warehouse for mobile inventory status accessible by both the OEMs and their distributors with data residing either on secure redundant servers or integrated within the OEMs' core business operating systems such as SAP or Oracle.

Among its many features, the system provides:
Writing of a unique serial number to each tagged device specific to each manufacturer's specifications;
Centralized visibility of consigned and loaned surgical inventory throughout the entire supply chain;
Real time access to each device's location and state of surgical readiness;
On-line link to product information, location history and device ownership/accountability.

The universal radio frequency tag technology utilized with the present invention is designed specifically for reusable medical devices (FIG. 2). Reference is hereby made to U.S. patent application Ser. No. 11/747,617, which is assigned to the assignee of the present invention and incorporated herein by reference. The tags 12 are durable and reusable to withstand the harsh rigors of sterilization and life cycle logistic processes. Implementing tags helps to automate tracking, creates unique identification, and improves control of valuable medical devices.

The RFID tag innovations are unique in that they withstand repeated harsh chemical decontamination and high heat sterilization, and they are small because they use the metal content of the asset as an antenna, thereby eliminating the space required for internal antennae. Using the asset as the antenna also enhances passive read distances. The high performance RFID tags are readable on metal at distances measured in feet rather than inches. The medical grade tags are durable under high heat, thereby withstanding repeated autoclave cycles and are biocompatible to ensure safety. Further, the RFID tags comply with global RFID UHF transmission standards, meaning its RFID tags integrate seamlessly with existing industry standard RFID readers.

The tags are designed for maximum flexibility and are adaptable to a wide range of medical devices, including surgical implants, instruments, and cases. The tags are small and available in multiple sizes and may be embedded in or attached to medical devices, such as surgical case 18.

The RFID tag designs fulfill the auto-identification visibility requirement of the system. These innovative proprietary medical grade tags are:

1. Designed to survive repeated sterile reprocessing (up to 1000 cycles);

2. Embeddable in or on metal and offer enhanced read distance and read rates;

3. Biocompatible for use in Class I, II and III FDA regulated surgical devices;

4. Small in size yet have extended read distance range in feet verses inches;

5. Customizable to accommodate hundreds of thousands of different surgical device designs; and

6. Manufactured cost-effectively and exclusively by established component level electronics manufacturers.

UHF RFID label inlays that don't require autoclaving or performance around metal are specified for packaged devices designed to work optimally within all the read locations within the distribution network. There are hundreds of various tag configurations available that are designed specifically for various factors that influence tag read performance, such as water, metal and heat.

Another type of reader or read location that may be used with the system 10 of the present invention is an RFID “smart cabinet” 16 (FIG. 4), which delivers a breakthrough in asset visibility, security, and automated inventory management. The smart cabinets use on-demand sensing to identify items removed or added to the cabinet, while transactions are automatically associated to the user. The smart cabinets provide fast, auto-inventory functionality for reliable management of valuable medical assets.

Reliable and secure inventory control features include the following:
Automatic check in/out provides 100% accountability
Quickly scans hundreds of tagged items simultaneously
E-mail notification of “out of stock” or “low stock” inventory
Auto-notification of an item's expiration, calibration date, etc.
Reduces inventory shrinkage and obsolescence
Improves product regulatory surveillance

The comprehensive versatility of the smart cabinets allows for data synchronization between multiple cabinets and existing company inventory systems. Additional features include web-based browser capabilities for remote inventory monitoring and reporting, an easy-to-use touch screen interface, and secured accessibility using access code, fingerprint, or smart pass.

Reader portals are configurable to a specific location such as a doorway or attached to a shelf that holds inventory. The reads captured by these hardware configurations are managed similar to reads captured in the smart cabinet.

| Location | Objective | | Procedure | |
|---|---|---|---|---|
| OEM | 1. | Assign a unique ID | 1. | An RFID tag with a unique identifier will be |
| manufacturing, | | (RFID) to case, | | affixed to the product. |
| kitting | | instrument or implant | 2. | Order information will be exported from |
| | | product | | OEM's ERP system and imported to |
| | 2. | Associate the unique ID | | proprietary software scan engine to be |
| | | of the product with the | | associated with the unique ID |
| | | order number and critical | 3. | Populate database that is common to all |
| | | order details | | read locations, with the unique ID and order |
| | 3. | Initiate location tracking | | information of the OEM |
| | | of tagged product | | |
| OEM | 1. | Confirm ship date and | 1. | The unique ID will be captured by a fixed |
| manufacturing, | | time | | read location as the product is transported |
| shipping | | | | to the shipping dock, and the Product's |
| | | | | status in the common database will be |
| | | | | updated. |
| Distributors 1&2 | 1. | Inbound: Update | 1. | The Product's unique ID will be read by a |
| | | distributor inventory to | | fixed read location as it is received by the |
| | | include additional | | distributor, and the common database will |
| | | product as product is | | be updated to include the Product in the |
| | | received into its facility | | distributor's inventory (FIG. 3) |
| | 2. | Outbound: Update | 2. | The Product's unique ID will be read by a |
| | | distributor inventory to | | fixed read location as it is dispatched from |
| | | remove product as they | | the distributor's facilities. The common |
| | | are removed from facility | | database will be updated to remove the |
| | 3. | Associate destination of | | Product from the distributor's inventory. |
| | | product in database with | | |
| | | interface provided to the | | |
| | | material mover | | |
| Hospitals 1-6 | 1. | Provide accurate | 1. | Product will be checked in and out of |
| | | hospital location | | hospital inventory utilizing a mobile |
| | | inventory information as | | handheld reader or a stationary reader, and |
| | | Products are moved | | the common database will be updated |
| | | into, and out of, facility | | accordingly |
| | | (FIG. 3) | 2. | Inventory information will be available on- |
| | 2. | Increase efficiency of | | demand through a web interface accessible |
| | | hospital inventory audit | | from internet-connected computers for all |
| | | process and regulatory | | locations |
| | | compliance | | |

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A system for identification, tracking and management of medical assets, comprising:
a plurality of radio frequency identification (RFID) tagged medical assets; and
a completely networked supply chain, including:
at least one end user location;
at least one distributor location;
an offsite data management system;
a plurality of RFID readers, each said reader being interspersed at a respective location throughout said networked supply chain and configured to electronically read data from said plurality of RFID tagged medical assets; and
at least one data network interconnecting each of said at least one end user location, said at least one distributor location, and said offsite data management system, said at least one data network providing real time information from at least one of said plurality of RFID readers to said offsite data management system concerning a current attribute associated with at least one of said RFID tagged medical assets.

2. The system of claim 1, wherein said current attribute associated with said at least one RFID tagged medical asset includes:
a current location,
a current time associated with the current location,
a unique identification number associated with a particular medical asset,
a manufacturing date,
an expiration date,
a calibration date,
a sterilization status,
a number of sterilization cycles,
a low stock indicator,
an out of stock indicator, and
a number of times a medical asset has been used in a surgical procedure.

3. The system of claim 1, wherein at least one of said RFID tagged medical assets utilize an RFID tag that is one of mounted to and integrated within a metal substrate of the medical asset and is tuned to use the metal substrate as an antenna for increased read distances.

4. The system of claim 1, wherein said at least one data network includes at least one of a local area network (LAN), a wide area network (WAN) and a cellular telephone connection.

5. The system of claim 4 wherein said LAN includes at least one of a wireless network and a wired network, and said WAN includes at least one of a wired network and an internet based network.

6. The system of claim 1, wherein said plurality of RFID readers include at least one of a cabinet with integral reader, a fixed portal reader, and a portable handheld reader.

7. The system of claim 6, wherein at least some of said plurality of RFID readers are positioned at predefined “choke points” in said supply chain to capture movement of said RFID tagged medical assets.

8. The system of claim 1, wherein at least one said end user location is a hospital.

9. The system of claim 1, wherein at least one said distributor location is one of a distributor warehouse and a sales representative vehicle.

10. The system of claim 1, wherein said medical assets comprise at least one of orthopaedic implants, surgical trays, surgical cases, and surgical instruments.

11. The system of claim 10, wherein each of said RFID tagged medical assets includes at least one RFID tag, each of said RFID tags being repeatedly sterilizable.
